(19) United States
(12) Patent Application Publication
(10) Pub. No.: US 2022/0370689 A1
(43) Pub. Date: Nov. 24, 2022

(54) URINARY CATHETERS AND METHODS FOR PREVENTING BACTERIAL INFECTIONS

(71) Applicant: Hollister Incorporated, Libertyville, IL (US)

(72) Inventors: Tadas S. Sileika, Lake Forest, IL (US); Nicole L. Dean, Antioch, IL (US); Jacqueline MJ Pettigrew, Lindenhurst, IL (US)

(21) Appl. No.: 17/771,716

(22) PCT Filed: Oct. 27, 2020
(86) PCT No.: PCT/US2020/057535

§ 371 Date: Apr. 25, 2022

Related U.S. Application Data
(60) Provisional application No. 62/926,923, filed on Oct. 28, 2019.

(51) Int. Cl. A61L 29/08; A01N 43/24; A61L 29/16

(52) U.S. Cl. CPC A61L 29/085; A01N 43/24; A61L 29/16; A61L 2300/406; A61L 2400/10

(57) ABSTRACT

Urinary catheters and methods for preventing bacterial infections.

The present application claims the benefit of and priority to U.S. Provisional Patent Application No. 62/926,923, filed Oct. 28, 2019, which is hereby incorporated herein by reference.

### DESCRIPTION

### Technical Field

The present disclosure generally relates to urinary catheters and methods for preventing bacterial infections, and more particularly, to catheters and methods for preventing urinary tract infections. The present disclosure also relates to urinary catheters and methods that hinder the motility structures of bacteria within the urethra.

### Background

It is desirable for urinary catheters to have a lubricated or lubricious outer surface to increase comfort of the patient during insertion into and/or removal from the body. One method for rendering the surface of a urinary catheter lubricious is to coat at least the insertable portion of the catheter with a lubricious hydrophilic coating. Another method is to apply a lubricant to the urinary catheter. Lubricity of the catheter minimizes soft tissue damage and reduces overall discomfort during use of the medical device.

Although catheters are typically prepared in sterile environments and are provided with safe handling instructions, catheter users are at risk of contracting a urinary tract infection due to several different factors. For example, the catheter may become contaminated during use and introduce bacteria into the urethra. The catheter also may carrier bacteria along the urinary tract. Additionally, because urine is voided from the bladder through the catheter, urine does not flow directly through the urethra. Thus, urine is no longer a factor in flushing out the urethra or wetting of the urethral tissue.

Therefore, there is a need for catheters which reduce the risk of urinary tract infections.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a urinary catheter product includes a catheter and an agent that hinders the motility structures of bacteria.

### BRIEF DESCRIPTION OF THE DRAWINGS

FIG. 1 is a plan view of one embodiment of the present disclosure; and

FIG. 2 is a plan view of one embodiment of a catheter of the present disclosure.

### DETAILED DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

For catheter users, whether in-dwelling or intermittent, urine is no longer a factor that participates in regular flushing or wetting of the urethra. Because the users are unable to void directly through the urethra, the urethra no longer experiences the continuous flushing and re-wetting of the tissues that normally occurs during active voluntary urination. When the mechanism fails, the risk of urinary tract infections increases.

Urinary tract infections can be a result of bacteria swarming and/or traveling up through the urethra towards the bladder. Traveling of bacteria up the urethra is typically not a concern for those who can perform voluntary urination, because normal voiding tends to wash out and eliminate the bacteria from urethra, thereby prohibiting it from traveling toward the bladder and causing infection.

There are certain species of bacteria that are considered good “swimmers,” which swarm and move when they receive certain signals from the surrounding environment. These bacteria are capable of movement even on solid surfaces through excretion of their own extracellular fluids. These motile bacteria also are known to become more pathogenic and increase their motility organelles, such as flagella (a process called hyperflagellation), when sensing certain signals, such as decreased viscosity in the fluids and mucus in their surrounding environment and/or an increase in chemotactants.

In a non-voiding patient, the urethral environment will be fairly viscous, due to the presence of mucosal secretions. When a hydrophilic catheter or a catheter lubricated with a water-based gel is introduced into the urethra, such introduction will likely deposit additional moisture, diluting the secretions, thus decreasing the overall viscosity of the urethral mucus and the surrounding urethral environment. This decrease in the urethral mucus viscosity provides a signal that activates the motility of the bacteria.

Furthermore, when a catheter is inserted into the urethra, the catheter, hydrophilic coating, hydration medium and/or lubricants may include a chemotactant that promotes chemotaxis (the movement of the bacteria in response to a chemical stimulant). For example, the hydrophilic coating and hydration medium may include a chemotactant, such as a polyol or polysaccharide, which stimulates the bacteria to move toward the chemotactant. If the catheter distributes this chemotactant at or near the bladder, the bacteria may be stimulated to undesirably swarm and move toward the bladder.

The catheter products disclosed herein target and hinder the mobility structures of the bacteria, such as the flagella and pili of the bacteria. The flagella and pili are some of the structures of the bacteria that are responsible for mobility. The catheter products of the present disclosure include agents that target and hinder, inhibit or disable the flagella and/or pili from performing their mobility functions. Hindering the flagella and/or pili from performing their functions, mitigates the risk of bacteria swarming and traveling proximally up the urethra toward the bladder. Thus, when bacteria are stimulated to swarm and move by signals from changes in the urethral environment, the bacteria do not effectively move and/or swarm because the agent has hindered the motility structure of the bacteria. The agents may be incorporated into the catheter product in a manner that the agents are delivered and/or distributed into the urethra by the catheter product.

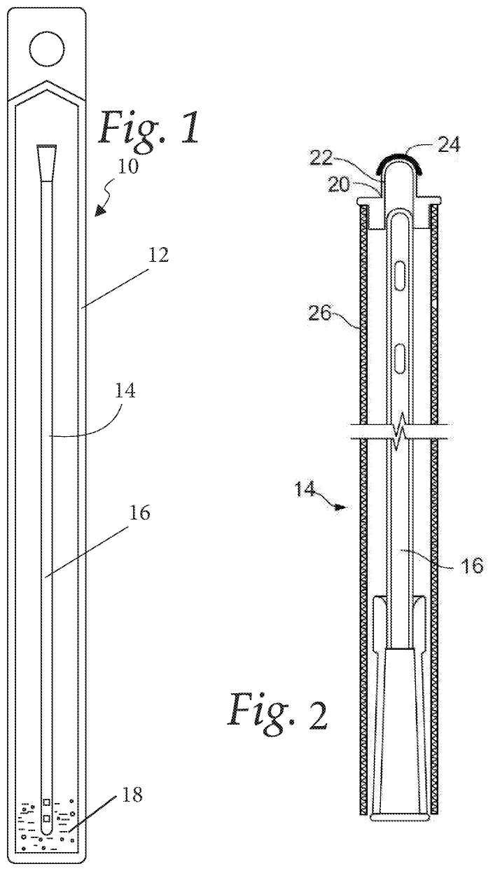

The agent that inhibits motility structures of bacteria may be included in any component of a catheter product. For example, referring to FIG. 1, a catheter product 10 includes a package 12 including a catheter 14 having a catheter shaft 16. The outer surface of the catheter shaft 16 may be hydrophilic. For example, the catheter 16 may be coated with a hydrophilic coating. The hydrophilic surface of the catheter shaft 16 becomes lubricious when wetted with a hydration medium 18 (water or saline). In one alternative, the agent that inhibits motility structures of the bacteria may be contained in the hydrophilic material (e.g., the hydrophilic coating) and/or the hydration medium. When the catheter product includes a lubricating gel, the compound may be included in the lubricating gel. When the catheter is inserted into and through the urethra, the agent may be distributed along the urethra, where it will come into contact with bacteria.

Referring to FIG. 2, when the catheter product includes an introducer aid 20 that may be inserted into the urethral opening and which the catheter is inserted through, the introducer aid 20 or the tip 22 thereof may include the agent that inhibits motility structures of bacteria 24. When the catheter shaft 16 is inserted through the introducer aid 20, the agent may be disposed onto the catheter shaft 16 wherein the catheter distributes the agent along the urethra. Additionally, a sleeve 26 may be attached to the introducer aid, wherein the sleeve surrounds the catheter.

In one embodiment, the agent downregulates gene expression associated with motility structures of bacteria. For example, the agent may suppress the expression of the flagellin and/or pili gene. Moreover, the agent or agents can target flagellar proteins, such as the C-ring, f-T3SS, motor-force generators, MS-ring, rod, hook, and filament. Additionally, the agent can target injectisome proteins, such as v-T3SS, MS-ring, C-ring, Secretin, Rod, Needle, and Translocon. Furthermore, the agent or agents can target T4P proteins, such as the retraction ATPase, assembly ATPase, pre-pilin peptidase, pre-pilin, pilin, secretin, and Type-IV Pilus. In addition or alternative to, the agent may decrease the bacteria's production of motility structures.

The agent may include pomegranate rind extract and/or polyphenolic molecules from pomegranate, which target and inhibit flagella of bacteria, thereby inhibiting the motility and swarming of the bacteria. In one embodiment, the agent may include punicalagin. In another embodiment, the agent may include a peptide compound that suppresses gene expressions associated with motility structures of bacteria. In yet another embodiment, the agent may include: branched-chain fatty acids, hydroxyindoles, naphthalene derivatives, quinazoline-2,4-diamino analogs, salicylaldehyde hydrazones, salicylidene anilines, and thiazolidinones.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

What is claimed is:

1. A urinary catheter product, comprising:
a catheter; and
an agent that hinders motility structures of bacteria.

2. The urinary catheter product of claim 1, wherein the catheter further includes a shaft and a hydrophilic coating on the shaft, and the agent being contained in the hydrophilic coating.

3. The urinary catheter product of claim 1, wherein the catheter further includes a shaft and a hydrophilic coating, and the agent being contained in a hydration medium for hydrating the hydrophilic coating.

4. The urinary catheter product of claim 1, further including a lubricant for lubricating the catheter, wherein the lubricant includes the agent.

5. The urinary catheter product of claim 1, wherein the agent downregulates gene expression associated with motility structures of bacteria.

6. The urinary catheter product of claim 1, wherein the agent decreases production of motility structures of bacteria.

7. The urinary catheter product of claim 1, wherein the agent suppresses the expression of a flagellin gene.

8. The urinary catheter product of claim 1, wherein the agent decreases flagella production.

9. The urinary catheter product of claim 1, wherein the agent suppresses the expression of a pili gene.

10. The urinary catheter product of claim 1, wherein the agent decreases pili production.

11. The urinary catheter product of claim 1, wherein the agent comprises pomegranate rind extract.

12. The urinary catheter product of claim 1, wherein the agent comprises polyphenolic molecules from pomegranate.

13. The urinary catheter product of claim 1, wherein the agent comprises punicalagin.

14. The urinary catheter product of claim 1, wherein the agent comprises a peptide compound that suppresses gene expressions associated with motility structures of bacteria.

15. The urinary catheter product of claim 1, wherein the agent comprises branched-chain fatty acids, hydroxyindoles, naphthalene derivatives, quinazoline-2,4-diamino analogs, salicylaldehyde hydrazones, salicylidene anilines, and thiazolidinones.
